(19) United States
(12) Patent Application Publication
(10) Pub. No.: US 2024/0139263 A1
(43) Pub. Date: May 2, 2024

(54) SCREENING PROBIOTIC STRAINS FOR IN VITRO GAS PRODUCTION TO IDENTIFY STRAINS THAT REDUCE IN VIVO (HUMAN) BLOATING AND RELATED CONDITIONS

(71) Applicant: Synbiotic Health, Inc., Lincoln, NE (US)

(72) Inventors: Zachery Lewis, American Fork, UT (US); Thomas Auchtung, Lincoln, NE (US); Mallory Van Haute, Gretna, NE (US)

(21) Appl. No.: 18/495,366

(22) Filed: Oct. 26, 2023

Related U.S. Application Data
(60) Provisional application No. 63/381,120, filed on Oct. 26, 2022.

(51) Int. Cl. A61K 35/747; A61K 35/745; A61K 47/26; A61P 1/14

(52) U.S. Cl. CPC A61K 35/747; A61K 35/745; A61K 47/26; A61P 1/14; C12R 2001/23

(57) ABSTRACT

Provided herein are methods and compositions for a probiotic composition comprising at least one probiotic strain selected based on gas production resulting from digestion of a specific food, food product, food ingredient or food component to reduce the amount of gas and or bloating produced when a subject consumes the specific food, food product, food ingredient, or food component.

### RELATED APPLICATIONS

This application claims priority to Application Ser. No. 63/381,120, filed Oct. 26, 2022, and entitled “Screening Probiotic Strains for in vitro gas production to identify strains that reduce in vivo (human) Bloating and Related Conditions,” the entire contents of which are incorporated herein by reference.

### TECHNICAL FIELD

This disclosure relates to methods of identifying probiotics that reduce the production of gas in the human gastrointestinal tract, including compositions containing probiotics and at least one additive.

### BACKGROUND

Excessive gas in the gastrointestinal tract can cause multiple symptoms, including bloating (abdominal swelling), belching, flatulence, cramps, rumbling, borborygmus, abdominal pain, nausea and vomiting, diarrhea, constipation, and/or loss of appetite. Approximately 15% to 30% of adults in the United States suffer from bloating. Non-US populations are reported to have similar levels of bloating.

Many different ingestible items can cause gas, bloating, diarrhea, and related issues, but the most common cause is the inability to properly digest dietary compounds, such as fermentable oligosaccharides, disaccharides, monosaccharides, and polyols (FODMAPs). As is known in the art, the consumption of certain foods lead to the production of gas and/or bloating. The amount of gas production and/or bloating from each food product consumed varies between individuals, even if the individuals are related or consume the same quantity of commercial probiotic product.

Microorganisms that inhabit the gastrointestinal tract produce various amounts of gas when metabolizing compounds that are in the food that has been consumed and passed through the gastrointestinal tract. The amount of gas produced depends on the specific dietary compounds that are being degraded and the organisms that are present and metabolizing the compounds. Numerous clinical studies have examined the effect of probiotics on gas production, with a wide degree of variation in improvement of symptoms.

In vitro models of human gastrointestinal gas production have been developed to measure the effect of adding different prebiotics. Some studies have examined how the abundance of specific fecal bacteria changed in the presence of prebiotics, however, they did not test whether gas production was changed by adding specific bacteria.

Accordingly, there is a need for methods of identifying probiotics and probiotic-containing compositions that can reduce the amount of gas produced by an individual after consuming a specific type of food, food product, or food by-product. There is a further need for probiotic and probiotic-containing compositions. Additionally, there remains a need for methods of administering and dosing such probiotics and probiotic-containing compositions in order to reduce the amount of gas produced by individuals consuming the food, food product, or food by-product.

### SUMMARY

Provided herein are methods and compositions for a probiotic strain selected by examining the ability of strains to reduce the production of gas associated with metabolism of dietary compounds under simulated gastrointestinal conditions. Additionally, compositions comprising the probiotic strain are also provided herein.

The methods and compositions are set forth in part in the description which follows, and in part will be obvious from the description, or can be learned by practice of the methods, apparatuses, and systems. The advantages of the methods and compositions will be realized and attained by means of the elements and combinations particularly pointed out in the appended claims. It is to be understood that both the foregoing general description and the following detailed description are exemplary and explanatory only and are not restrictive of the methods, apparatuses, and systems, as claimed.

Accordingly, it is an object of the disclosure not to encompass within the disclosure any previously known product, process of making the product, or method of using the product such that Applicants reserve the right and hereby disclose a disclaimer of any previously known product, process, or method. It is further noted that the disclosure does not intend to encompass within the scope of the disclosure any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the disclosure to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved.

In one aspect of the present disclosure, a composition to reduce gas production and/or bloating is provided. In some forms, the composition is formulated to reduce gas and/or bloating after eating a certain type of food, a certain item of food, and/or a specific component or ingredient of a food product. In some forms, the composition includes a quantity of a probiotic. In some forms, the composition includes a probiotic and a prebiotic. In some forms, the prebiotic and the component or ingredient of the food is the same. In some forms, the composition includes more than 1, 2, 3, 4, 5, 6, 7, 8, 9, 10, 11, 12, 13, 14, 15, 16, 17, 18, 19, and/or 20 probiotics. In some forms, the composition includes more than 1, 2, 3, 4, 5, 6, 7, 8, 9, 10, 11, 12, 13, 14, 15, 16, 17, 18, 19, and/or 20 prebiotics. In some embodiments, the probiotic is selected from the group consisting of Lactobacillus, Bifidobacterium, Bacillus, Streptococcus, Aspergillus, Streptomyces Saccharomyces, Enterococcus, Turicibacter, Veillonellla, Anaerostipes, Catenibacterium, Mediterraneibacter, Prevotella, and any combination thereof. In some forms, the probiotic is a species or strain of a genus selected from the group consisting of Lactobacillus, Bifidobacterium, Bacillus, Streptococcus, Aspergillus, Streptomyces Saccharomyces, Enterococcus, Turicibacter, Veillonellla, Anaerostipes, Catenibacterium, Mediterraneibacter, Prevotella, and any combination thereof. In some forms, the probiotic is selected from the group consisting of Bifidobacterium adolescentis, Bifidobacterium animalis subsp. lactis, Bifidobacterium animalis subsp. animalis, Bifidobacterium bifidum, Bifidobacterium dentium, Bifidobacterium longum subsp. longum, Bifidobacterium longum subsp. infantis, Bifidobacterium longum subsp. suillum, Bifidobacterium catenulatum, Bifidobacterium pseudocatenulatum, Bifidobacterium kashiwanohense, Bifidobacterium faecale, Bifidobacterium breve, Bifidobacterium thermoacidophilum subsp. porcinum, Bifidobacterium thermophilum, Lactobacillus acidophilus, Lactobacillus gasseri, Lactobacillus crispatus, Ligilactobacillus animalis, Ligilactobacillus ruminis, Lactiplantibacillus plantarum, Lacticaseibacillus rhamnosus, Lacticaseibacillus paracasei, Limosilactobacillus vaginalis, and any combination thereof. In some embodiments, the component, ingredient of food, or prebiotic is selected from the group consisting of fructooligosaccharides, isomaltulose, xylooligosaccharides, galactooligosaccharides, cyclodextrins, raffinose, oligosaccharides, soybean oligosaccharides, lactulose, lactosucrose, maltooligosaccharides, isomaltooligosaccharides, arabinoxylooligosaccharides, enzyme resistant dextrin, glucooligosaccharides, oligofructose, lactose, mannan-oligosaccharides, fructans, inulin, transgalactooligosaccharides, levans, polydextrose, D-tagatose, and any combination thereof. In some embodiments, the food that causes the gas and/or bloating is selected from the group consisting of beans, lentils, vegetables such as asparagus, broccoli, brussels sprouts, cabbage, artichokes, and onions, wheat, dairy products such as milk, yogurt, ice cream, and cheese, fruits such as apples, peaches, pears, and prunes, oat bran, peas, and other foods high in soluble fiber, corn, pasta, potatoes, and other foods rich in starch, whole grains, such as brown rice, oatmeal, and whole wheat, and any combination thereof.

In some embodiments, the probiotic interacts with and/or supports enzymes that are present in the digestive tract when contacting a specific prebiotic, food, component of food, ingredient of food, or any combination thereof. In some forms, presence of the probiotic composition in the gastrointestinal tract results in less gas production, bloating, and any combination thereof when encountering a certain type of food, food product, food ingredient, food byproduct, or food component found in food. Preferably, the reduction in gas production and/or bloating is in comparison to the amount of gas produced or bloating if the probiotic or probiotic composition is not present in the gastrointestinal tract. In some forms, the probiotic or probiotic composition is present in the gastrointestinal tract after administration of the probiotic or probiotic composition to an animal in need thereof. In some forms, the amount of gas produced is reduced by 5, 10, 15, 20, 25, 30, 35, 40, 45, 50, 55, 60, 65, 70, 75, 80, 85, 90, 95% or more when the probiotic is present than when the probiotic is not present. In some forms, the probiotic or probiotic is administered to an animal in need thereof prior to or at the same time as ingesting a food, food component, or specific food ingredient. In some forms, the probiotic or probiotic composition is administered at least 5, 10, 15, 20, 30, 40, 50, 60, 70, 80, 90, 100, 110, 120, 130, 140, 150, 160, 170, 180, 210, 240, 270, 300, 360, 420, 480, 540, 600, 660, 720, 780, or more minutes prior to the ingestion of the food, food product, food ingredient, food byproduct, or food component is consumed. In some forms, the probiotic or probiotic composition is administered on a regular basis. In some forms, the probiotic or probiotic composition is administered at least 1, 2, 3, 4, 5, 6, 7, 8, 9, 10 or more times a day at least 1, 2, 3, 4, 5, 6, or seven days per week.

In some forms, the probiotic or probiotic composition further includes at least one digestive enzyme. Examples of suitable digestive enzymes may include, but are not limited to, an enzyme that performs the first step in breaking down complex carbohydrates, or enzymes that perform separate functions not performed efficiently by probiotics. In some aspects, the digestive enzyme may include, but are not limited to, inulinase, lactase, or a combination thereof.

In another aspect, the present disclosure provides methods for the selection of probiotics that produce less gas when consuming a specific food, food product, food ingredient, or food component. In general, the specific food, food product, food ingredient, or food component is contacted with a media formulated to simulate the composition found in the gut. Any conventional gut media formulation will work for purposes of the present disclosure. Such media generally includes Peptone (0.2-1.0 g/L), Yeast Extract (0.25-2.0 g/L), Sodium Chloride (0.25-2.0 g/L), Magnesium Sulfate Heptahydrate (0.05-0.2 g/L), Calcium Chloride (0.05-0.2 g/L), Hemin (0.00005-0.001 g/L), Cysteine HCl (0.2-1.0 g/L), Bile salts (0.2-1.0 g/L), Sodium acetate (0.5-3 g/L), Sodium propionate (0.2-0.7 g/L), Isobutyric acid (0.2-1 mM), Isovaleric acid (0.2-1 mM), Valeric acid (0.2-1 mM), Sodium bicarbonate (2-6 g/L), Potassium phosphate (0.2-1 g/L), Vitamin K1 (0.3-2 mg/L), Maltose (0.05-0.2 g/L), Cellobiose (0.05-0.2 g/L), Inulin (0.05-0.2 g/L), Arabinogalactan (0.05-0.2 g/L), Soluble starch (0.05-0.2 g/L), buffer (pH 7.0-7.6) (25-300 mM). One preferred formulation of the gut media comprises Peptone 0.5 g/L, Yeast Extract 1.0 g/L, Sodium Chloride 1.0 g/L, Magnesium Sulfate Heptahydrate 0.1 g/L, Calcium Chloride 0.1 g/L, Hemin 0.0005 g/L, Cysteine HCl 0.5 g/L, Bile salts 0.5 g/L, Sodium acetate 1.35 g/L, Sodium propionate 0.43 g/L, Isobutyric acid 0.5 mM, Isovaleric acid 0.5 mM, Valeric acid 0.5 mM, Sodium bicarbonate 4 g/L, Potassium phosphate 0.45 g/L, Vitamin K1 1 mg/L, Maltose 0.1 g/L, Cellobiose 0.1 g/L, Inulin 0.1 g/L, Arabinogalactan 0.1 g/L, Soluble starch 0.1 g/L, and MOPS buffer pH 7.3 100 mM.

In general, the method comprises the step of establishing a simulated gastrointestinal environment by adding an amount of gut media to a container. In preferred forms, the container will be sealable in order to prevent the unmeasured escape of gas from the container. In some forms, the containers are Hungate tubes or autosampler vials. Hungate tubes, as well as many other suitable containers, can be sealed with a stopper. In some forms, the stopper is further secured in place, for example by crimping an aluminum seal over the stopper. When autosampler vials are used, they have a seal effected through a screwable lid or top. The containers are further adapted to permit the sampling of gas produced by the gut media in combination with the food, food product, food ingredient, or food component, and probiotic. In some forms, the containers include a septa through which gas sampling can occur. In some forms, the septa is formed using a material that permits the insertion of a syringe and/or needle to sample or withdraw gas. In some forms, the septa is formed of silica and/or PTFE. The amount of gut media can be in any amount sufficient for the gut media and food, food product, food ingredient, or food component, and an amount of probiotic. In some forms, the amount of gut media ranges from 0.25 ml to more than 250 ml. When Hungate tubes are used, the amount of gut media is closer to 25 ml. When autosampler vials are used, the amount of gut media used is closer to 0.5 ml. Preferably, the composition of this gut media resembles that of the contents of the gastrointestinal tract, particularly the colon where most problems with excessive gas are thought to occur. It typically contains peptides, bile salts, short chain fatty acids, low levels of complex carbohydrates, and a strong buffer. Preferred gut media formulations are provided above. Communities of fecal microorganisms are added by diluting frozen fecal samples with a frozen fecal sample to gut media ratio of at least 1:50 (i.e., at least 1:75, 1:100, 1:125, 1:150, 1:175, 1:200, 1:250, 1:300, 1:350, 1:400, 1:450, 1:500, and more). In some embodiments, the frozen fecal sample may be further diluted with a buffer, such as, for example, phosphate-buffered saline (PBS). In preferred forms, the methods are performed under anoxic conditions. In some forms, the methods are performed in an anaerobic chamber with 90% N₂, 5% CO₂, and 5% H₂ atmosphere.

Prior to establishing the simulated gastrointestinal environment, individual strains are prepared by growing in rich media, pelleting cells by centrifugation, resuspending in gut media containing cryoprotectant, and freezing.

To identify which probiotics are best at treating a specific gas-causing compound in a food for reducing gas production, an individual compound such as a prebiotic, food, food product, food ingredient, or food component is added to the gut media, typically at a final concentration of between about 0.5 wt-% to about 5 wt-% in the gut media. Potential probiotic strains are added to individual Hungate tubes at equal cell concentrations.

In order to prevent loss of gas produced by the community, stoppers are inserted into the top of the Hungate tubes, then an aluminum seal is crimped over it to keep it in place. After a set time of incubation with shaking, the amount of gas is measured by inserting a needle attached to a syringe through the stopper. The gas pushes the plunger of the syringe, allowing measurement of the volume of gas released.

### BRIEF DESCRIPTION OF THE DRAWINGS

FIG. 1 is a graph showing the amount of gas produced by fecal communities grown in gut media containing Compound 1 (sorbitol) plus one of thirteen candidate probiotics.

FIG. 2 is a graph showing the amount of gas produced by fecal communities grown in gut media containing Compound 2 (raffinose) plus one of thirteen candidate probiotics.

FIG. 3 is a graph showing the amount of gas produced by fecal communities grown in gut media containing 5% garbanzo beans plus one of thirteen candidate probiotics.

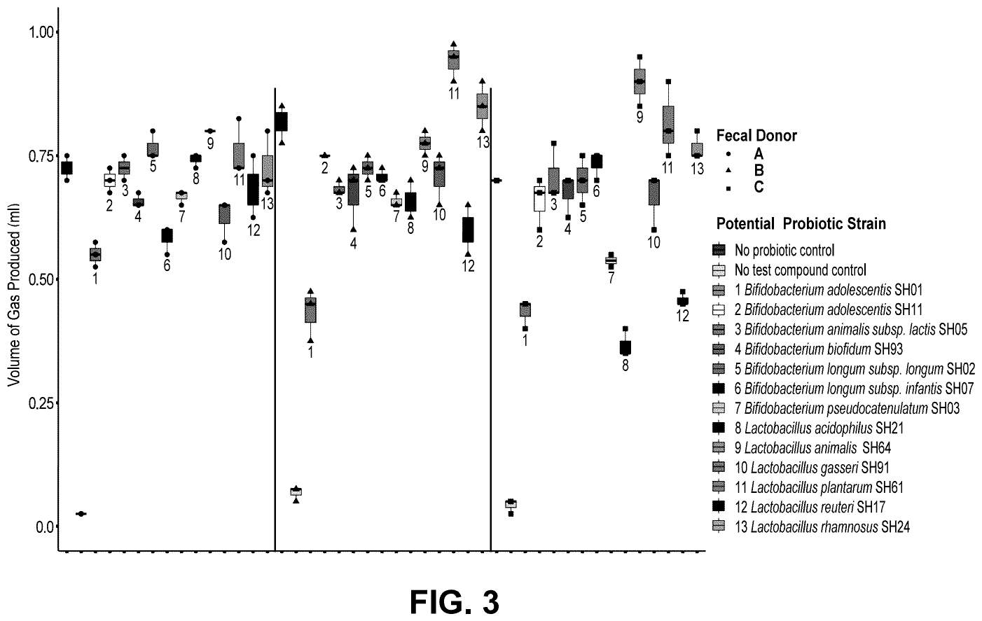

Various embodiments of the present disclosure will be described in detail with reference to the figures. Reference to various embodiments does not limit the scope of the disclosure. Figures represented herein are not limitations to the various embodiments according to the disclosure and are presented for exemplary illustration of the disclosure.

### DETAILED DESCRIPTION

The foregoing and other features and advantages of the disclosure are apparent from the following detailed description of exemplary embodiments, read in conjunction with the accompanying drawings. The detailed description and drawings are merely illustrative of the disclosure rather than limiting, the scope of the disclosure being defined by the appended claims and equivalents thereof.

Embodiments of the disclosure will now be described with reference to the Figures. The terminology used in the description presented herein is not intended to be interpreted in any limited or restrictive way, simply because it is being utilized in conjunction with detailed description of certain specific embodiments of the disclosure. Furthermore, embodiments of the disclosure may include several novel features, no single one of which is solely responsible for its desirable attributes or which is essential to practicing the disclosures described herein.

The use of the terms “a” and “an” and “the” and similar referents in the context of describing the disclosure are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the disclosure and does not pose a limitation on the scope of the disclosure unless otherwise claimed. No language in the specification should be construed as indicating any nonclaimed element as essential to the practice of the disclosure.

References to “one embodiment,” “an embodiment,” “example embodiment,” “various embodiments,” etc., may indicate that the embodiment(s) of the disclosure so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### Definitions

By the expression “probiotics” is referred a composition which comprises probiotic microorganisms. Probiotic bacteria are defined as live bacteria, which when administered in adequate amounts confer a health benefit on the host. Probiotic microorganisms have been defined as “Live microorganisms which when administered in adequate amounts confer a health benefit on the host” (FAO/WHO 2002).

By the expression “prebiotic” is referred to a composition or a component of a composition which is selectively utilized by host microorganisms conferring a health benefit”. Prebiotics are generally non-viable food components that are specifically fermented in the colon by bacteria thought to be of positive value, e.g. bifidobacteria, lactobacilli, and other short-chain fatty acid producing microorganisms. Prebiotics are also known as an “ingredient that allows specific changes, both in the composition and/or activity in the gastrointestinal microbiota that confers benefits upon host well-being and health” (see, for example, Roberfroid, 1998, Br. J. Nutr., 80:S197-202). The combined administration of a probiotic strain with one or more prebiotic compounds, when designed optimally, may enhance the growth of the administered probiotic in vivo resulting in additional or more pronounced health benefits, and is termed a synbiotic. A synbiotic is formally defined as “a mixture, comprising live microorganisms and substrate(s) selectively utilized by host microorganisms, that confers a health benefit on the host”. Well characterized prebiotics include, for example, galactooligosaccharides (GOS), fructooligosaccharides (FOS), and inulin. GOS and FOS refer to a group of oligomeric, non-digestible carbohydrates that are often produced from monomers using glycosidases to catalyze transgalactosylation reactions. These carbohydrates are often recalcitrant to digestion by host-secreted enzymes in the small intestine, such that they reach the colon intact and are available to the colonic microbiota. It would be understood by those skilled in the art that other compounds that fall within the definition of a prebiotic also can be used in the methods described herein.

As used herein, a “subject” or “individual” or “animal” can refer to a human or a non-human. Representative non-human subjects include, without limitation, livestock (e.g., swine, cow, horse, goat, and sheep), poultry (e.g., fowls such as chicken and turkey), and companion animals (e.g., pets such as dogs and cats).

The food, food component, or food by-product of the present disclosure can be any ingestible material selected from the group consisting of fructooligosaccharides, isomaltulose, xylooligosaccharides, galactooligosaccharides, cyclodextrins, raffinose, oligosaccharides, soybean oligosaccharides, lactulose, lactosucrose, maltooligosaccharides, isomaltooligosaccharides, arabinoxylooligosaccharides, enzyme resistant dextrin, glucooligosaccharides, oligofructose, inulin, transgalactooligosaccharides, levans, polydextrose, D-tagatose, and any combination thereof. In some forms, the food that causes the gas and/or bloating is selected from the group consisting of beans, lentils, vegetables such as asparagus, broccoli, brussels sprouts, cabbage, artichokes, and onions, wheat, dairy products such as milk, curd, milk based fermented products, acidified milk, yoghurt, frozen yoghurt, milk powder, milk based powders, milk concentrate, cheese, cheese spreads, ice-creams, infant formula, fruits such as apples, peaches, pears, and prunes, oat bran, peas, and other foods high in soluble fiber, corn, pasta, potatoes, and other foods rich in starch, whole grains, such as brown rice, oatmeal, and whole wheat, and any combination thereof.

In a further embodiment, the composition further comprises a pharmaceutically acceptable carrier. As used herein, the term “pharmaceutically acceptable carrier” means one or more solid or liquid filler diluents or encapsulating substances which are suitable for administration to a human or an animal and which is/are compatible with the active probiotic organisms. The term “compatible” relates to components of the pharmaceutical composition which are capable of being comingled with a probiotic bacteria or a mutant strain thereof in a manner enabling no interaction that would substantially reduce the probiotic efficacy of the organisms selected for the disclosure under ordinary use conditions. Pharmaceutically acceptable carriers must be of a sufficiently high purity and a sufficiently low toxicity to render them suitable for administration to humans and animals being treated.

A bacterial “strain” as used herein refers to a bacterium which remains genetically unchanged when grown or multiplied. The multiplicity of identical bacteria is included. “Wild type strain” refers to the non-mutated form of a bacterium, as found in nature. In the present context, the term “derived strain” should be understood as a strain derived from a mother strain by means of e.g. genetic engineering, normal laboratory and commercial production culturing, radiation and/or chemical treatment, and/or selection, adaptation, screening, etc. In specific embodiments the derived strain is a functionally equivalent mutant, e.g. a mutant that has substantially the same, or improved, properties (e.g. regarding probiotic properties) as the mother strain. Such a derived strain is a part of the present disclosure. The term “derived strain” includes a strain obtained by subjecting a strain of the disclosure to any conventionally used mutagenization treatment including treatment with a chemical mutagen such as ethane methane sulphonate (EMS) or N-methyl-N′-nitro-N-nitroguanidine (NTG), UV light or to a spontaneously occurring mutant.

A “mutant bacterium” or a “mutant strain” refers to a natural (spontaneous, naturally occurring) mutant bacterium or an induced mutant bacterium comprising one or more mutations in its genome (DNA) which are absent in the wild type DNA. An “induced mutant” is a bacterium where the mutation was induced by human treatment, such as treatment with any conventionally used mutagenization treatment including treatment with chemical mutagens, such as a chemical mutagen selected from (i) a mutagen that associates with or become incorporated into DNA such as a base analogue, e.g. 2-aminopurine or an interchelating agent such as ICR-191, (ii) a mutagen that reacts with the DNA including alkylating agents such as nitrosoguanidine or hydroxylamine, or ethane methyl sulphonate (EMS) or N-methyl-N′-nitro-N-nitroguanidine (NTG), UV- or gamma radiation etc. In contrast, a “spontaneous mutant” or “naturally occurring mutant” has not been mutagenized by man. A derived strain, such as a mutant, may have been subjected to several mutagenization treatments (a single treatment should be understood one mutagenization step followed by a screening/selection step), but typically no more than 20, no more than 10, or no more than 5, treatments are carried out. In specific embodiments of derived strains, such as mutants, less than 1%, less than 0.1%, less than 0.01%, less than 0.001% or even less than 0.0001% of the nucleotides in the bacterial genome have been changed (such as by replacement, insertion, deletion or a combination thereof) compared to the mother strain. Mutant bacteria as described above are non-GMO, i.e. not modified by recombinant DNA technology. As an alternative to above preferred method of providing the mutant by random mutagenesis, it is also possible to provide such a mutant by site-directed mutagenesis, e.g. by using appropriately designed PCR techniques or by using a transposable element which is integratable in bacterial replicons.

When the mutant is provided as a spontaneously occurring mutant the above wild-type strain is subjected to the selection step without any preceding mutagenization treatment.

In one embodiment, a solid composition as described herein is a tablet, a capsule or a granulate (comprising a number of granules), or a powder. Preferably the solid composition is an oral dosage form. A review of conventional formulation techniques can be found in e.g., Lachman, Leon., Liebermann, Herbert A. The Theory and Practice of Industrial Pharmacy. India: CBS Publishers & Distributors Pvt. Limited, 2009. Thus, the tablets may be prepared by methods known in the art and can be compressed, enterically coated, sugar coated, film coated, or multiply compressed, containing suitable binders, lubricants, diluents, disintegrating agents, coloring agents, flavoring agents, flow-inducing agents, and melting agents. Capsules, both soft and hard capsules, having liquid or solid contents, may be prepared according to conventional techniques that are well known in the pharmaceutical industry. As one example, the active probiotic organisms may be filled into gelatine capsules, using a suitable filling machine. A solid composition as described herein may also be a pellet or granule. In one embodiment, oral delivery via dry, not hot foods and supplements. An acceptable carrier needs to be used to standardize the strain concentrations for appropriate dosing. In further embodiments, the composition may be a liquid suspension, a paste, or a syrup.

In some forms, the probiotic or probiotic composition further includes at least one additive selected from the group consisting of preservatives, solvents, dispersion media, coatings, stabilizing agents, antifungal agents, isotonic agents, adsorption delaying agents, and any combination thereof.

The human or animal food composition or dosage form should comprise at least one probiotic or probiotic strain or a mutant strain thereof, as described above, so that the amount of the probiotic strain that is available for the individual is between about 10³-10¹⁴ CFU per day, such as between about 106-10¹³ CFU per day, including between about 108-10¹² CFU per day, or even between about 109-10¹³ CFU per day. The amount of probiotic strain depends on the individual weight of the subject, and it is preferably of between about 109-10¹² CFU/day for humans and between about 10⁷-10¹² CFU/day for pets. It will be understood, however, that the specific dose level for any particular subject will depend upon a variety of factors including the activity of the specific compound employed, the community structure and content of the microbiota, the age, body weight, general health, sex, diet, time of administration, route of administration, rate of excretion, formula combination, and the severity of the particular target condition of the subject.

In some forms, the probiotic or probiotic strain is from a genus selected from the group consisting of Lactobacillus, Bifidobacterium, Bacillus, Streptococcus, Aspergillus, Streptomyces Saccharomyces, Enterococcus, Turicibacter, Veillonellla, Anaerostipes, Catenibacterium, Mediterraneibacter, Prevotella, and any combination thereof. In some forms, the probiotic or probiotic strain is selected from the group consisting of Bifidobacterium adolescentis, Bifidobacterium animalis subsp. lactis, Bifidobacterium animalis subsp. animalis, Bifidobacterium bifidum, Bifidobacterium dentium, Bifidobacterium longum subsp. longum, Bifidobacterium longum subsp. infantis, Bifidobacterium longum subsp. suillum, Bifidobacterium catenulatum, Bifidobacterium pseudocatenulatum, Bifidobacterium kashiwanohense, Bifidobacterium faecale, Bifidobacterium breve, Bifidobacterium thermoacidophilum subsp. porcinum, Bifidobacterium thermophilum, Lactobacillus acidophilus, Lactobacillus gasseri, Lactobacillus crispatus, Ligilactobacillus animalis, Ligilactobacillus ruminis, Lactiplantibacillus plantarum, Lacticaseibacillus rhamnosus, Lacticaseibacillus paracasei, Limosilactobacillus vaginalis, and any combination thereof.

In a further embodiment, the compositions of the present disclosure, including but not limited to a human or pet food composition or dietary supplement dosage form, further comprises one or more prebiotic substances. Examples of suitable prebiotic substances are selected from the group consisting of fructooligosaccharides, isomaltulose, xylooligosaccharides, galactooligosaccharides, cyclodextrins, raffinose, oligosaccharides, soybean oligosaccharides, lactulose, lactosucrose, lactose, mannan-oligosaccharides, fructans, maltooligosaccharides, isomaltooligosaccharides, arabinoxylooligosaccharides, enzyme resistant dextrin, glucooligosaccharides, oligofructose, inulin, transgalactooligosaccharides, levans, polydextrose, D-tagatose, and any combination thereof.

In some forms, the probiotic or probiotic composition is combined with or consumed concurrently with a food that causes the gas and/or bloating. In some forms, the food is selected from the group consisting of beans, lentils, vegetables such as asparagus, broccoli, brussels sprouts, cabbage, artichokes, and onions, wheat, dairy products such as milk, yogurt, ice cream, and cheese, fruits such as apples, peaches, pears, and prunes, oat bran, peas, and other foods high in soluble fiber, corn, pasta, potatoes, and other foods rich in starch, whole grains, such as brown rice, oatmeal, and whole wheat, and any combination thereof. In some forms, the probiotic or probiotic composition is used as a top-dressing to a food. In some forms, the probiotic or probiotic composition is mixed with the food. In some forms, the probiotic is mixed with the food during the preparation thereof. Preferably, when combined with the food, the temperature of the food during the production or serving thereof does not impact the viability of the probiotic.

### Reduction of Gas

The disclosure further provides methods of decreasing the production of gas caused by a food, food product, food by-product or any combination thereof. This decreased gas production preferably persists for at least 0.25, 0.5, 1, 2, 3, 4, 5, 6, 7, 8, 9, 10, 11, 12, 13, 14, 15, 16, 17, 18, 19, 20, 21, 22, 23, 24, 30, 36, 42, 48, 60, 72, 84, 96 hours or more after administration of the probiotic or probiotic composition. In some forms, the decreased gas production lasts for more than 1, 2, 3, 4, or more weeks, 1, 2, 3, 4, 5, 6, 7, 8, 9, 10, 11, 12, or more months, and any combination thereof after the administration of the probiotic or probiotic composition.

Individuals who may benefit from the methods and compositions of the disclosure include individuals suffering from the symptoms of gas production and/or bloating after the consumption of a food, food product, food ingredient, or food component, as described above. Any degree of gas production may be treated or prevented by the methods of the disclosure. Symptoms of gas production include, but are not limited to, gas, bloating, abdominal discomfort, diarrhea, and/or cramping. Effectiveness of treatment may be measured in a number of ways. Conventional measurements, such as hydrogen gas production, symptom surveys, dietary recall surveys, and/or microbiome measures may be used before and after treatment. Alternatively, or in addition, the amount of the food, food product, food ingredient, or food component that may be consumed before the onset of one or more symptoms may be measured or evaluated before and after treatment, thus, for example, treatment is considered at least partially effective if, after treatment, on average less gas is produced with a given dose of the probiotic or probiotic composition after consumption of the food, food product, food ingredient, or food component.

Alternatively, individuals will not precisely test the amount of gas produced to measure effectiveness. Instead, individuals generally have a sense of how much of the food, food product, food ingredient, or food component they may consume, and the types and degree of symptoms experienced after such consumption.

As used herein, “partial” elimination of symptoms of gas production is a statistically significant increase in the amount of a food, food product, food ingredient, or food component that may be consumed before the onset of symptoms, or a statistically significant (P<0.05) reduction in reporting of some symptoms in digestive surveys. “Substantial” elimination of symptoms of gas production, as used herein, encompasses the above effects where the result is both statistically and clinically significant, as measured by appropriate effect size statistical evaluation. “Complete” or “substantially complete” elimination of symptoms of gas production, as used herein, indicates that normal amounts of the food, food product, food ingredient, or food component may be consumed after treatment (i.e., the amount of such product in a typical diet for the area and/or culture in which the individual normally lives) without symptoms, or with only the rare occurrence of symptoms. Thus, for example, an individual may know that if he or she consumes one half cup of beans that there will be no, or minimal, symptoms, but if 1 or more cup of beans is consumed, then symptoms such as gas or bloating occur. The individual may find that, after treatment, 1 and one-half cups of beans may be consumed but that 3 or more cups cause symptoms, this indicates that symptoms of gas production were substantially eliminated. Alternatively, the individual may find that after treatment, a normal diet for their geographical or cultural region may be consumed with no, or rare, symptoms of gas production. In that case, symptoms of gas production were completely eliminated.

More specifically, effectiveness may be measured by percent decrease in symptoms of gas production. In this measurement, the severity of a predetermined symptom, or set of symptoms is measured before and after treatment, e.g., using a pre and post Likert scale. Exemplary symptoms for measurement include gas, bloating, and abdominal pain. Any one, or more than one, of the symptoms may be measured. For example, an individual may be asked to rate one or more symptoms on a scale of increasing severity from 1 to 5. In one embodiment, a set of symptoms is rated, and the ratings are added; for example, gas, bloating, and abdominal pain may be rated. The percentage decrease in symptoms from before treatment to after treatment may be calculated, and the symptoms of gas production may be considered eliminated by that percent decrease (e.g., if there is a 50% decrease in symptoms, then symptoms of gas production is 50% eliminated).

In some embodiments, the disclosure provides a method of decreasing symptoms of gas production in an individual exhibiting symptoms of excess gas production by administering to the individual increasing amounts of a specific food, food product, food ingredient, or food component for a period of time, wherein one or more symptoms of gas production are partially, substantially, or completely eliminated. In some embodiments, the symptom(s) of gas production remains partially, substantially, or completely eliminated for at least about 1 day, 1 week, 1 month, 2 months, 3 months, 4 months, 5 months, 6 months, 9 months, one year, 18 months, two years, three years, four years, five years, or more than five years after the termination of treatment, or permanently after the termination of treatment. In some embodiments, the disclosure provides a method of decreasing symptoms of gas production in an individual exhibiting symptoms of excess gas production by administering to the individual increasing amounts of a specific food, food product, food ingredient, or food component for a period of time, wherein symptoms of gas production are substantially eliminated for at least about one month after treatment is terminated.

In some embodiments, the disclosure provides a method of decreasing symptoms of gas production in a subject exhibiting symptoms of excess gas production by administering to the subject at least one probiotic as described herein, and increasing amounts of a specific food, food product, food ingredient, or food component for a period of time, wherein the symptoms of gas production, measured as described herein, are decreased by an average of about 10%, 20%, 30%, 40%, 50%, 60%, 70%, 80%, 90%, 95%, or about 100%. As used herein, an “average” decrease is a decrease as measured in a group of individuals exhibiting symptoms of gas production, such as more than about 2, 3, 4, 5, 10, 25, or 50 individuals. In some embodiments, the decrease of symptoms of gas production persists or becomes even greater (e.g., 50% decrease to 55% decrease) for at least about 1 day, 1 week, 1 month, 2 months, 3 months, 4 months, 5 months, 6 months, 9 months, one year, 18 months, two years, three years, four years, five years, or more than five years after the termination of treatment. In some embodiments, the decrease in symptoms is permanent. In some embodiments, the disclosure provides a method of decreasing symptoms of gas production in an individual exhibiting symptoms of excess gas production by administering to the individual at least one probiotic as described herein, and increasing amounts of a specific food, food product, food ingredient, or food component for a period of time, wherein the symptoms of gas production, measured as described herein, are decreased by an average of about least about 20% and remain decreased by at least about 20% for at least about one month after treatment is terminated. In some embodiments, the disclosure provides a method of decreasing symptoms of gas production in an individual exhibiting symptoms of excess gas production by administering to the individual at least one probiotic as described herein, and increasing amounts of the food, food product, food ingredient, or food component for a period of time, wherein the symptoms of gas production, measured as described herein, are decreased by an average of about least about 50% and remain decreased by at least about 50% for at least about one month after treatment is terminated.

### Duration of Treatment

The total duration of treatment may be from about 1 or more days, weeks, months, years, and any combination thereof. During this period of time, the subject is started on a program of treatment, and subsequently challenged with a food, food product, food ingredient, or food component that caused symptoms of gas or excess gas production prior to the treatment. In some embodiments the treatment comprises at least one administration of a probiotic or probiotic composition as described herein. It will be appreciated that these durations are averages, and that individuals using the treatment may vary from the average based on the severity of their symptoms, their existing microbiome content, missing days of treatment, and the like. In some embodiments, the duration of the treatment is based on the individual's symptoms or existing microbiome content. Thus, an individual may experience a return of symptoms at a given intake of the food, food product, food ingredient, or food component, and may require an adjustment of treatment dose and/or time, or a lowering their intake of the food, food product, food ingredient, or food component, until symptoms subside. Thus, in some embodiments, the duration of the treatment is not definitively established at the outset, but continues until the highest dose of the food, food product, food ingredient or food component is achieved, or until the desired level of gas reduction is achieved.

Increasing dosage of the probiotic or probiotic composition may be achieved by increasing the amount of the probiotic included in the dose administered, or by increasing the number of doses, or both. In one embodiment, both strategies are used. Thus, in some embodiments of the disclosure, the probiotic or probiotic composition is initially administered at a certain dosage until the symptoms of gas production are reduced or eliminated, followed by a reduction of that dosage until symptoms resume, upon which time, the dosage is increased to just above the level that permitted symptoms to resume. This process can be repeated multiple times. Preferably, the treatment will result in the colonization of the gastrointestinal tract by the probiotic that produces less gas when consuming the food, food product, food ingredient, or food component such that continued treatment becomes unnecessary.

After the treatment has concluded, the individual is encouraged to enjoy the food, food product, food ingredient, or food component regularly, such as every other day, twice a week, or once a week in order to maintain colonization by the probiotic strain that reduced the gas production.

### DESCRIPTION OF EMBODIMENTS

A composition or a foodstuff that includes the probiotic and the prebiotic as described herein can further include a pharmaceutically acceptable carrier. A pharmaceutically acceptable carrier should be non-toxic to the bacteria and to the animal, and also can include an ingredient that promotes viability of the microorganism during storage. Liquid or gel-based carriers are well known in the art, such as water, fruit juice, glucose or fructose solutions, physiological electrolyte solutions, and glycols such as methanol, ethanol, propanol, butanol, ethylene glycol and propylene glycol. Carriers also include oleaginous carriers such as, for example, white petrolatum, isopropyl myristate, lanolin or lanolin alcohols, mineral oil, fragrant or essential oil, nasturtium extract oil, sorbitan mono-oleate, cetylstearyl alcohol, hydroxypropyl cellulose (MW=100,000 to 1,000,000), or detergents (e.g., polyoxyl stearate or sodium lauryl sulfate). Other suitable carriers include water-in-oil or oil-in-water emulsions and mixtures of emulsifiers and emollients are provided.

A composition or a foodstuff that includes the probiotic and the prebiotic, as described herein, also can include natural or synthetic flavorings and food-quality coloring agents, thickening agents such as corn starch, guar gum, xanthan gum and the like, binders, disintegrators, coating agents, lubricants, stabilizers, solubilizing agents, suspending agents, excipients, and diluents. Additional components also can be included that, for example, improve palatability, improve shelf-life, and impart nutritional benefits. It would be understood by those in the art that any additional components in a composition must be compatible with maintaining the viability of the microbial strain.

Administration of a composition or a foodstuff that includes the probiotic and the prebiotic as described herein can be accomplished by any method that delivers at least a portion of the probiotic and prebiotic into the digestive tract of an animal. Therefore, enteral administration is preferred (e.g., orally, sublingually, or rectally), although other routes are not excluded. Generally, the formulation of a composition is dependent upon its intended route of delivery. For example, a composition as described herein can be formulated as a powder, a granule, a tablet, a capsule, a liquid suspension, a paste, or a syrup.

In Example 1, a method of reducing gas and/or bloating caused by the digestion of a food, food product, food ingredient, or food component, the method comprises, administering a probiotic composition comprising at least one probiotic selected for its ability to digest said food, food product, food ingredient, or food component with a reduced amount of gas production in comparison to the amount of gas produced when said at least one probiotic is not administered, wherein said probiotic composition further comprises at least one additive selected from the group consisting of preservatives, solvents, dispersion media, coatings, stabilizing agents, antifungal agents, isotonic agents, adsorption delaying agents, and any combination thereof.

Example 2 relates to the method according to Example 1, wherein the at least one probiotic is from a genus selected from the group consisting of Lactobacillus, Bifidobacterium, Bacillus, Streptococcus, Aspergillus, Streptomyces Saccharomyces, Enterococcus, Turicibacter, Veillonellla, Anaerostipes, Catenibacterium, Mediterraneibacter, Prevotella, and any combination thereof.

Example 3 relates to the method according to Example 1, wherein the at least one probiotic is selected from the group consisting of Bifidobacterium adolescentis, Bifidobacterium animalis subsp. lactis, Bifidobacterium animalis subsp. animalis, Bifidobacterium bifidum, Bifidobacterium dentium, Bifidobacterium longum subsp. longum, Bifidobacterium longum subsp. infantis, Bifidobacterium longum subsp. suillum, Bifidobacterium catenulatum, Bifidobacterium pseudocatenulatum, Bifidobacterium kashiwanohense, Bifidobacterium faecale, Bifidobacterium breve, Bifidobacterium thermoacidophilum subsp. porcinum, Bifidobacterium thermophilum, Lactobacillus acidophilus, Lactobacillus gasseri, Lactobacillus crispatus, Ligilactobacillus animalis, Ligilactobacillus ruminis, Lactiplantibacillus plantarum, Lacticaseibacillus rhamnosus, Lacticaseibacillus paracasei, Limosilactobacillus vaginalis, and any combination thereof.

Example 4 relates to the method according to Example 1, wherein said food, food product, food ingredient, or food component is selected from the group consisting of fructooligosaccharides, isomaltulose, xylooligosaccharides, galactooligosaccharides, cyclodextrins, raffinose, oligosaccharides, soybean oligosaccharides, lactulose, lactosucrose, maltooligosaccharides, isomaltooligosaccharides, arabinoxylooligosaccharides, enzyme resistant dextrin, glucooligosaccharides, oligofructose, lactose, mannan-oligosaccharides, fructans, inulin, transgalactooligosaccharides, levans, polydextrose, D-tagatose, beans, lentils, vegetables, artichokes, onions, pears wheat, dairy products, fruits, oat bran, foods high in soluble fiber, foods rich in starch, whole grains, and any combination thereof.

Example 5 relates to the method according to Example 1, wherein said gas and/or bloating is reduced after administering said probiotic composition at least one time to a subject in need thereof.

Example 6 relates to the method according to Example 1, wherein said probiotic is present in the probiotic composition in an amount of at least 10³ CFU.

Example 7 relates to the method according to Example 1, wherein said probiotic composition is administered orally.

Example 8 relates to the method according to Example 1, wherein said probiotic composition further comprises an effective amount of the food, food product, food ingredient, or food component for reducing gas and/or bloating.

Example 9 relates to the method according to Example 1, wherein the amount of gas produced after the administration is reduced by at least 10% in comparison to the amount of gas produced after consumption of the same food, food product, food ingredient, or food component without the administration of the probiotic composition.

Example 10 relates to the method according to Example 1, wherein said food, food product, food ingredient, or food component comprises sorbitol and said probiotic comprises Bifidobacterium pseudocatenulatum SH03.

Example 11 relates to the method according to Example 1, wherein said food, food product, food ingredient, or food component comprises raffinose and said probiotic comprises Bifidobacterium adolescentis SH01.

In Example 12, a composition for reducing gas production and/or bloating in a subject comprises a probiotic comprising Bifidobacterium adolescentis SH01 or Bifidobacterium pseudocatenulatum SH03; and at least one additive selected from the group consisting of preservatives, solvents, dispersion media, coatings, stabilizing agents, antifungal agents, isotonic agents, adsorption delaying agents, and any combination thereof, wherein the composition reduces the amount of gas produced by the digestion of raffinose or sorbitol.

Example 13 relates to the composition according to Example 12, wherein the probiotic is the Bifidobacterium adolescentis SH01 and is present in the probiotic composition in an amount of at least 10³ CFU.

Example 14 relates to the composition according to Example 13, wherein said composition further comprises an amount of raffinose.

Example 15 relates to the composition according to Example 12, wherein said composition is formulated for oral administration.

Example 16 relates to the composition according to Example 12, wherein the probiotic is Bifidobacterium pseudocatenulatum SH03 and is present in the composition in an amount of at least 10³ CFU.

Example 17 relates to the composition according to Example 16, wherein said composition further comprises an amount of sorbitol.

In Example 18, a composition for reducing the production of gas and/or bloating in the gastrointestinal tract after the consumption of at least one food, food product, food ingredient, or food component, the composition comprises, at least one probiotic selected from a genus consisting of Lactobacillus, Bifidobacterium, Bacillus, Streptococcus, Aspergillus, Streptomyces Saccharomyces, Enterococcus, Turicibacter, Veillonellla, Anaerostipes, Catenibacterium, Mediterraneibacter, Prevotella, and any combination thereof; and at least one additive selected from the group consisting of preservatives, solvents, dispersion media, coatings, stabilizing agents, antifungal agents, isotonic agents, adsorption delaying agents, and any combination thereof, wherein the at least one food, food product, food ingredient, or food component is selected from the group consisting of fructooligosaccharides, isomaltulose, xylooligosaccharides, galactooligosaccharides, cyclodextrins, raffinose, oligosaccharides, soybean oligosaccharides, lactulose, lactosucrose, maltooligosaccharides, isomaltooligosaccharides, arabinoxylooligosaccharides, enzyme resistant dextrin, glucooligosaccharides, oligofructose, lactose, mannan-oligosaccharides, fructans, inulin, transgalactooligosaccharides, levans, polydextrose, D-tagatose, beans, lentils, vegetables, artichokes, onions, wheat, dairy products, fruits, oat bran, foods high in soluble fiber, foods rich in starch, whole grains, and any combination thereof.

Example 19 relates to the composition according to Example 18, further comprising an effective amount of said at least one food, food product, food ingredient, or food component for reducing the production of gas and/or bloating.

Example 20 relates to the composition according to Example 18, wherein said at least one probiotic comprises Bifidobacterium adolescentis SH01, Bifidobacterium longum subsp. infantis SH07, Bifidobacterium pseudocatenulatum SH03, Lactobacillus acidophilus SH21, Lactobacillus gasseri SH91, Lactobacillus reuteri SH17, or a combination thereof.

All publications and patent applications in this specification are indicative of the level of ordinary skill in the art to which this disclosure pertains. All publications and patent applications are herein incorporated by reference to the same extent as if each individual publication or patent application was specifically and individually indicated as incorporated by reference.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as the disclosure. However, those of skill in the art should, in light of the present disclosure, appreciate that many changes can be made in the specific embodiments which are disclosed and still obtain a like or similar result without departing from the spirit and scope of the disclosure.

Efforts have been made to ensure accuracy with respect to numbers (e.g., amounts, temperature, etc.), but some errors and deviations should be accounted for. Unless indicated otherwise, parts are parts by weight, temperature is in ° C., or is at ambient temperature, and pressure is at or near atmospheric.

### Example 1

### Selection of Strains that Produce Less Gas from a Given Substrate

5 ml of gut media was added to 270 25 ml Hungate tubes. The composition of this gut media was peptone 0.5 g/L, yeast extract 1.0 g/L, sodium chloride 1.0 g/L, magnesium sulfate heptahydrate 0.1 g/L, calcium chloride 0.1 g/L, hemin 0.0005 g/L, cysteine HCl 0.5 g/L, bile salts 0.5 g/L, sodium acetate 1.35 g/L, sodium propionate 0.43 g/L, isobutyric acid 0.5 mM, isovaleric acid 0.5 mM, valeric acid 0.5 mM, sodium bicarbonate 4 g/L, potassium phosphate 0.45 g/L, vitamin K1 1 mg/L, maltose 0.1 g/L, cellobiose 0.1 g/L, inulin 0.1 g/L, arabinogalactan 0.1 g/L, soluble starch 0.1 g/L, and MOPS buffer pH 7.3 100 mM. Communities of fecal microorganisms from 3 different donors were added by diluting frozen fecal samples at 1:50 in the gut media.

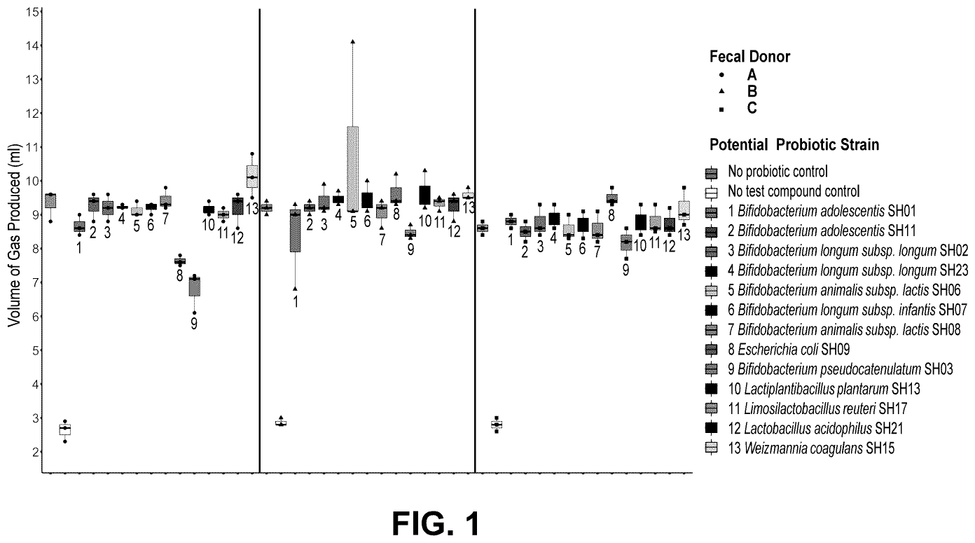

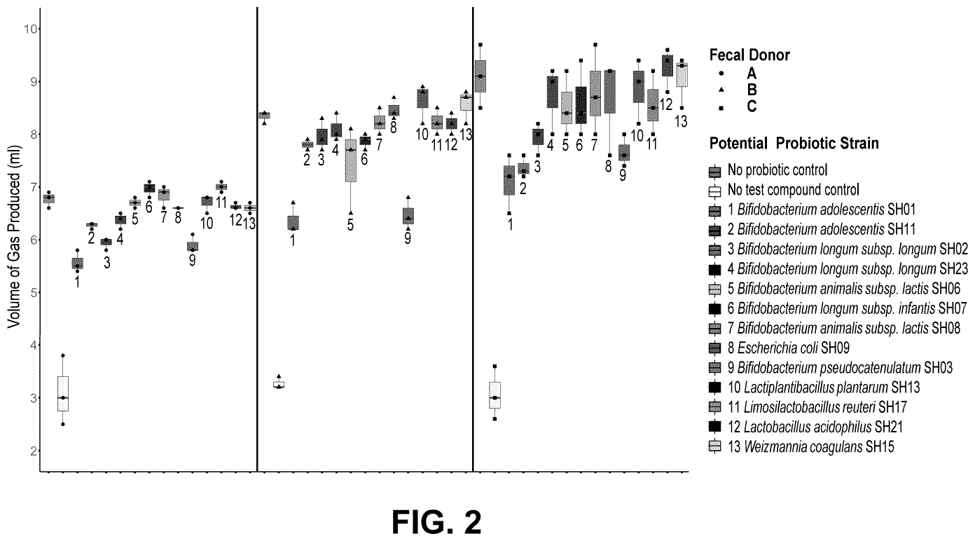

Prior to experiments, 13 individual probiotic strains were prepared by growing in rich media, pelleting cells by centrifugation, resuspending in gut media containing cryoprotectant, and freezing. To identify which probiotics were best at treating a specific compound, sorbitol was added to 14 of the Hungate tubes and raffinose was added to another 14 Hungate tubes. Each of these compounds was added to the gut media at 1% final concentration. Each of the probiotic strains prepared above was added at equal cell concentrations to two of the Hungate tubes, one of which included the raffinose and one of which included the sorbitol. In order to prevent loss of gas produced by the community, stoppers were inserted into the top of the Hungate tubes, then an aluminum seal was crimped over it to keep it in place. After a set time of incubation with shaking, the amount of gas was measured by inserting a needle attached to a syringe through the stopper. The gas pushed the plunger of the syringe, allowing measurement of the volume of gas released. Results of this experiment are provided in FIGS. 1 and 2. In those figures, Compound 1 was sorbitol and Compound 2 was raffinose. The strains used were as follows.

Strains (Genus, species, and strain number): 1 Bifidobacterium adolescentis SH01, 2 Bifidobacterium adolescentis SH11, 3 Bifidobacterium longum subsp. longum SH02, 4 Bifidobacterium longum subsp. longum SH23, 5 Bifidobacterium animalis subsp. lactis SH06, 6 Bifidobacterium longum subsp. infantis SH07, 7 Bifidobacterium animalis subsp. lactis SH08, 8 Escherichia coli SH09, 9 Bifidobacterium pseudocatenulatum SH03, 10 Lactiplantibacillus plantarum SH13, 11 Limosilactobacillus reuteri SH17, 12 Lactobacillus acidophilus SH21, and 13 Weizmannia coagulans SH15.

As can be seen, the amount of gas produced by the various combinations varied greatly with Bifidobacterium pseudocatenulatum SH03 producing the least amount of gas on average when combined with sorbitol (FIG. 1) and Bifidobacterium adolescentis SH01 producing the least amount of gas on average when combined with raffinose (FIG. 2).

### Example 2

### Selection of Strains that Produce Less Gas from a Given Food Substrate

0.5 ml gut media was added to 135 2 ml autosampler vials. The composition of this gut media was peptone 0.5 g/L, yeast extract 1.0 g/L, sodium chloride 1.0 g/L, magnesium sulfate heptahydrate 0.1 g/L, calcium chloride 0.1 g/L, hemin 0.0005 g/L, cysteine HCl 0.5 g/L, bile salts 0.5 g/L, sodium acetate 1.35 g/L, sodium propionate 0.43 g/L, isobutyric acid 0.5 mM, isovaleric acid 0.5 mM, valeric acid 0.5 mM, sodium bicarbonate 4 g/L, potassium phosphate 0.45 g/L, vitamin K1 1 mg/L, maltose 0.1 g/L, cellobiose 0.1 g/L, inulin 0.1 g/L, arabinogalactan 0.1 g/L, soluble starch 0.1 g/L, and MOPS buffer pH 7.3 100 mM. Communities of fecal microorganisms from at least 3 different donors was added by diluting frozen fecal samples at 1:500 in the gut media.

Prior to experiments, each of the individual probiotic strains were prepared by growing in rich media, pelleting cells by centrifugation, resuspending in gut media containing cryoprotectant, and freezing.

To identify which probiotics are best at treating specific gas-causing compounds in a food for reducing gas, 5% garbanzo beans was added to each of the autosampler vials. Each of the probiotic strains prepared above was added at equal cell concentrations to each of the autosampler vials. In order to prevent loss of gas produced by the community, lids were screwed tightly onto the top of the autosampler vials. After a set time of incubation with shaking, the amount of gas was measured by inserting a needle attached to a syringe through the stopper. The gas pushed the plunger of the syringe, allowing measurement of the volume of gas released. The strains used were as follows.

Strains (Genus, species, and strain number): 1 Bifidobacterium adolescentis SH01, 2 Bifidobacterium adolescentis SH11, 3 Bifidobacterium animalis subsp. lactis SH05, 4 Bifidobacterium bifidum SH93, 5 Bifidobacterium longum subsp. longum SH02, 6 Bifidobacterium longum subsp. infantis SH07, 7 Bifidobacterium pseudocatenulatum SH03, 8 Lactobacillus acidophilus SH21, 9 Lactobacillus animalis SH64, 10 Lactobacillus gasseri SH91, 11 Lactobacillus plantarum SH61, 12 Lactobacillus reuteri SH17, and 13 Lactobacillus rhamnosus SH24.

As can be seen, the amount of gas produced by the various combinations varied between the different probiotic strains. However, reduced gas was seen with a number of probiotic strains, including Bifidobacterium adolescentis SH01, Bifidobacterium longum subsp. infantis SH07, Bifidobacterium pseudocatenulatum SH03, Lactobacillus acidophilus SH21, Lactobacillus gasseri SH91, and Lactobacillus reuteri SH17. These combinations resulting in the least amount of gas produced may be selected for inclusion in a garbanzo bean composition.

While the disclosure has been described in connection with various embodiments, it will be understood that the disclosure is capable of further modifications. This application is intended to cover any variations, uses or adaptations of the disclosure following, in general, the principles of the disclosure, and including such departures from the present disclosure as, within the known and customary practice within the art to which the disclosure pertains.

What is claimed is:

1. A method of reducing gas and/or bloating caused by the digestion of a food, food product, food ingredient, or food component, the method comprising:
administering a probiotic composition comprising at least one probiotic selected for its ability to digest said food, food product, food ingredient, or food component with a reduced amount of gas production in comparison to the amount of gas produced when said at least one probiotic is not administered, wherein said probiotic composition further comprises at least one additive selected from the group consisting of preservatives, solvents, dispersion media, coatings, stabilizing agents, antifungal agents, isotonic agents, adsorption delaying agents, and any combination thereof.

2. The method of claim 1, wherein the at least one probiotic is from a genus selected from the group consisting of Lactobacillus, Bifidobacterium, Bacillus, Streptococcus, Aspergillus, Streptomyces Saccharomyces, Enterococcus, Turicibacter, Veillonellla, Anaerostipes, Catenibacterium, Mediterraneibacter, Prevotella, and any combination thereof.

3. The method of claim 1, wherein the at least one probiotic is selected from the group consisting of Bifidobacterium adolescentis, Bifidobacterium animalis subsp. lactis, Bifidobacterium animalis subsp. animalis, Bifidobacterium bifidum, Bifidobacterium dentium, Bifidobacterium longum subsp. longum, Bifidobacterium longum subsp. infantis, Bifidobacterium longum subsp. suillum, Bifidobacterium catenulatum, Bifidobacterium pseudocatenulatum, Bifidobacterium kashiwanohense, Bifidobacterium faecale, Bifidobacterium breve, Bifidobacterium thermoacidophilum subsp. porcinum, Bifidobacterium thermophilum, Lactobacillus acidophilus, Lactobacillus gasseri, Lactobacillus crispatus, Ligilactobacillus animalis, Ligilactobacillus ruminis, Lactiplantibacillus plantarum, Lacticaseibacillus rhamnosus, Lacticaseibacillus paracasei, Limosilactobacillus vaginalis, and any combination thereof.

4. The method of claim 1, wherein said food, food product, food ingredient, or food component is selected from the group consisting of fructooligosaccharides, isomaltulose, xylooligosaccharides, galactooligosaccharides, cyclodextrins, raffinose, oligosaccharides, soybean oligosaccharides, lactulose, lactosucrose, maltooligosaccharides, isomaltooligosaccharides, arabinoxylooligosaccharides, enzyme resistant dextrin, glucooligosaccharides, oligofructose, lactose, mannan-oligosaccharides, fructans, inulin, transgalactooligosaccharides, levans, polydextrose, D-tagatose, beans, lentils, vegetables, artichokes, onions, pears wheat, dairy products, fruits, oat bran, foods high in soluble fiber, foods rich in starch, whole grains, and any combination thereof.

5. The method of claim 1, wherein said gas and/or bloating is reduced after administering said probiotic composition at least one time to a subject in need thereof.

6. The method of claim 1, wherein said probiotic is present in the probiotic composition in an amount of at least 10³ CFU.

7. The method of claim 1, wherein said probiotic composition is administered orally.

8. The method of claim 1, wherein said probiotic composition further comprises an effective amount of the food, food product, food ingredient, or food component for reducing gas and/or bloating.

9. The method of claim 1, wherein the amount of gas produced after the administration is reduced by at least 10% in comparison to the amount of gas produced after consumption of the same food, food product, food ingredient, or food component without the administration of the probiotic composition.

10. The method of claim 1, wherein said food, food product, food ingredient, or food component comprises sorbitol and said probiotic comprises Bifidobacterium pseudocatenulatum SH03.

11. The method of claim 1, wherein said food, food product, food ingredient, or food component comprises raffinose and said probiotic comprises Bifidobacterium adolescentis SH01.

12. A composition for reducing gas production and/or bloating in a subject comprising:
a probiotic comprising Bifidobacterium adolescentis SH01 or Bifidobacterium pseudocatenulatum SH03; and
at least one additive selected from the group consisting of preservatives, solvents, dispersion media, coatings, stabilizing agents, antifungal agents, isotonic agents, adsorption delaying agents, and any combination thereof,
wherein the composition reduces the amount of gas produced by the digestion of raffinose or sorbitol.

13. The composition of claim 12, wherein the probiotic is the Bifidobacterium adolescentis SH01 and is present in the probiotic composition in an amount of at least 10³ CFU.

14. The composition of claim 13, wherein said composition further comprises an amount of raffinose.

15. The composition of claim 12, wherein said composition is formulated for oral administration.

16. The composition of claim 12, wherein the probiotic is Bifidobacterium pseudocatenulatum SH03 and is present in the composition in an amount of at least 10³ CFU.

17. The composition of claim 16, wherein said composition further comprises an amount of sorbitol.

18. A composition for reducing the production of gas and/or bloating in the gastrointestinal tract after the consumption of at least one food, food product, food ingredient, or food component, the composition comprising:
at least one probiotic selected from a genus consisting of Lactobacillus, Bifidobacterium, Bacillus, Streptococcus, Aspergillus, Streptomyces Saccharomyces, Enterococcus, Turicibacter, Veillonellla, Anaerostipes, Catenibacterium, Mediterraneibacter, Prevotella, and any combination thereof; and
at least one additive selected from the group consisting of preservatives, solvents, dispersion media, coatings, stabilizing agents, antifungal agents, isotonic agents, adsorption delaying agents, and any combination thereof,
wherein the at least one food, food product, food ingredient, or food component is selected from the group consisting of fructooligosaccharides, isomaltulose, xylooligosaccharides, galactooligosaccharides, cyclodextrins, raffinose, oligosaccharides, soybean oligosaccharides, lactulose, lactosucrose, maltooligosaccharides, isomaltooligosaccharides, arabinoxylooligosaccharides, enzyme resistant dextrin, glucooligosaccharides, oligofructose, lactose, mannan-oligosaccharides, fructans, inulin, transgalactooligosaccharides, levans, polydextrose, D-tagatose, beans, lentils, vegetables, artichokes, onions, wheat, dairy products, fruits, oat bran, foods high in soluble fiber, foods rich in starch, whole grains, and any combination thereof.

19. The composition of claim 18, further comprising an effective amount of said at least one food, food product, food ingredient, or food component for reducing the production of gas and/or bloating.

20. The composition of claim 18, wherein said at least one probiotic comprises Bifidobacterium adolescentis SH01, Bifidobacterium longum subsp. infantis SH07, Bifidobacterium pseudocatenulatum SH03, Lactobacillus acidophilus SH21, Lactobacillus gasseri SH91, Lactobacillus reuteri SH17, or a combination thereof.
